(19) United States
(12) Patent Application Publication
(10) Pub. No.: US 2020/0327981 A1
(43) Pub. Date: Oct. 15, 2020

(54) MEDICAL INFORMATION PROCESSING SYSTEM

(71) Applicant: Konica Minolta, Inc, Tokyo (JP)

(72) Inventor: Hiroaki MATSUMOTO, Yokohama-shi (JP)

(21) Appl. No.: 16/813,009

(22) Filed: Mar. 9, 2020

(30) Foreign Application Priority Data
Apr. 9, 2019 (JP) 2019-074034

(51) Int. Cl. G16H 40/20; G16H 30/40; G01R 33/54; G06T 7/00; G06T 11/00; G01R 33/28

(52) U.S. Cl. CPC G16H 40/20; G16H 30/40; G01R 33/543; G06T 7/0012; G06T 2207/20081; G01R 33/288; G06T 2207/10116; G06T 2207/10088; G06T 11/008

(57) ABSTRACT

A medical information processing system includes a first hardware processor that analyzes a medical image, in which the first hardware processor detects a metal contraindicated in MRI examinations from the medical image.

### CROSS-REFERENCE TO RELATED APPLICATIONS

The entire disclosure of Japanese Patent Application No. 2019-074034 filed on Apr. 9, 2019 is incorporated herein by reference in its entirety.

### BACKGROUND

### Technological Field

The present invention relates to a medical information processing system.

### Description of the Related Art

In recent years, in the medical field, image examination using magnetic resonance imaging (MRI) has been performed. The MRI is an apparatus for imaging the inside of the human body using nuclear magnetism. However, when a treatment tool formed of a predetermined metal, such as some endoscope clips or a pacemaker, is present inside the body (including the body surface) of a patient to be subjected to an examination, the treatment tool causes heating or artifacts in a captured image. For this reason, it is necessary to take measures, such as restricting MRI examination conditions, attendance of a doctor in the examination, and not performing the examination.

The MRI examination is performed by a laboratory technician according to the examination order from a doctor. At this time, the doctor or the laboratory technician checks whether or not there is any MRI contraindication based on examination information or medical records, such as the patients chief complaint or past examination history, to determine whether or not to perform the MRI examination. However, due to omission of the patients own notification or oversight of past examination history information, the MRI examination may be performed even though there is a metal, which is contraindicated in the MRI examination, in the body.

Therefore, for patients whose X-ray images or CT images have been captured in the past, the presence or absence of a metal contraindicated in MRI examinations is checked on these images on a day before the examination or the like. However, the visual image check imposes a heavy work burden on laboratory technicians or doctors.

As a technique for automatically detecting a metal from an X-ray image, for example, JP 2002-330953A discloses a technique of generating a cumulative histogram of pixel values of an X-ray image, setting, for example, 5% from the lower portion thereof as a pixel value representing a metal region, and detecting, as a metal region, pixels having pixel values equal to or less than the pixel value and pixels adjacent to the pixels at predetermined intervals therebetween.

### SUMMARY

However, artificial materials embedded in the body include materials other than metals that are contraindicated in MRI examinations. For example, a lead plate indicating the irradiation field aperture or the imaging direction in the X-ray image does not affect MRI examinations even though the lead plate is formed of a metal material contraindicated in MRI examinations, and accordingly, the lead plate is not contraindicated in MRI examinations. According to the technique disclosed in JP 2002-330953A, it is only necessary to uniformly detect a metal region, so that it is difficult to detect only metals contraindicated in MRI examinations. For this reason, in the case of determining whether or not to perform an MRI examination by detecting a metal region from an X-ray image using the technique disclosed in JP 2002-330953A, a metal structure or the like that is not contraindicated in the MRI examination is also detected. Therefore, it is necessary for the laboratory technician or the doctor to check whether or not the MRI examination can be performed with the metal.

It is an object of the present invention to accurately detect whether or not a metal contraindicated in MRI examinations is embedded in the body of a patient to be subjected to an MRI examination and to reduce the time and effort required for a laboratory technician or a doctor to check an image.

To achieve at least one of the abovementioned objects, according to an aspect of the present invention, a medical information processing system reflecting one aspect of the present invention is a medical information processing system including a first hardware processor that analyzes a medical image. The first hardware processor detects a metal contraindicated in MRI examinations from the medical image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a diagram illustrating the overall configuration of a medical information processing system according to an embodiment of the present invention;

FIG. 2 is a diagram schematically illustrating a procedure of detecting a metal contraindicated in MRI examinations from a medical image;

FIG. 3 is a flowchart illustrating an order list display process performed by a hardware processor of a medical information management apparatus illustrated in FIG. 1 in a first embodiment;

FIG. 4 is a flowchart illustrating an MRI examination order generation process performed by a hardware processor of an order generator illustrated in FIG. 1 in a third embodiment; and

FIG. 5 is a flowchart illustrating a checking process during imaging that is performed by a hardware processor of an X-ray imaging apparatus illustrated in FIG. 1 in a fourth embodiment.

### DETAILED DESCRIPTION OF THE EMBODIMENTS

Hereinafter, embodiments of the present invention will be described with reference to the diagrams. However, the scope of the present invention is not limited to the illustrated examples.

### First Embodiment

[Configuration of a Medical Information Processing System 100]

First, the configuration of a first embodiment will be described.

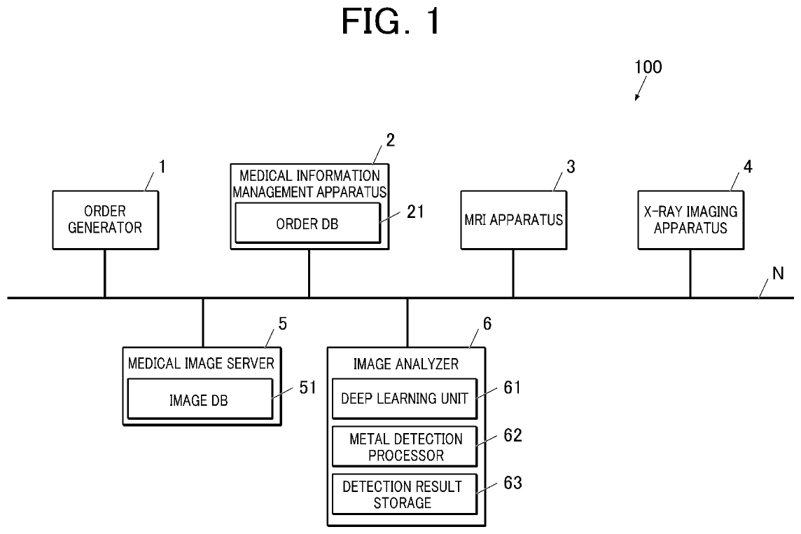

FIG. 1 illustrates the overall configuration of the medical information processing system 100 according to the first embodiment.

As illustrated in FIG. 1, the medical information processing system 100 is configured to include an order generator 1, a medical information management apparatus 2, an MRI apparatus 3, an X-ray imaging apparatus 4, a medical image server 5, an image analyzer 6, and the like. These apparatuses 1 to 6 are connected to each other through a communication network N constructed in a medical facility, such as a local area network (LAN), so as to be able to transmit and receive data to and from each other. For the communication network N, a digital imaging and communication in medicine (DICOM) standard is applied. The number of apparatuses is not particularly limited.

The order generator 1 is a computer apparatus including a hardware processor, a storage, an operation interface, a display, a communicator, and the like.

The hardware processor is configured by a central processing unit (CPU), a random access memory (RAM), and the like, and controls each unit of the order generator 1 by cooperation between the CPU and a program stored in the RAM to perform various kinds of processing. The storage is, for example, a hard disc drive (HDD), a semiconductor memory. The operation interface is a keyboard or a pointing device, such as a mouse, and the display is a liquid crystal display (LCD) or the like. The communicator is a LAN card or the like. A hardware processor, a storage, an operation interface, a display, and a communicator in each apparatus described below have the same configuration as described above.

The order generator 1 generates examination order information according to an input operation by an operator, and transmits the generated examination order information to the medical information management apparatus 2. As the order generator 1, for example, a hospital information system (HIS) can be applied.

The examination order information includes an order ID for identifying the examination order information, information of a patient as an examination target (patient ID, patient name, gender, age, and the like), and examination information (examination ID, examination date, requesting doctor name, image generation apparatus (modality), examination contents (examination part, laterality (left, right), direction, reservation procedure description, examination description, series description, and the like)). In the present application, patients include a person who is suspected of having a disease and needs to be examined.

The medical information management apparatus 2 is a computer apparatus including a hardware processor, a storage, an operation interface, a display, a communicator, and the like, and manages the examination order information transmitted from the order generator 1. For example, the medical information management apparatus 2 stores the examination order information transmitted from the order generator 1 in the an order database (DB) 21 of the storage, and displays examination order information that matches the search conditions input by the operator or transmits examination order information of the designated date or modality to the requesting apparatus in response to a request from another apparatus included in the medical information processing system 100. As the medical information management apparatus 2, for example, a radiology information system (RIS) can be applied.

The MRI apparatus 3 is configured to include a hardware processor, a storage, an operation interface, a display, a communicator, an image generator, and the like. The image generator of the MRI apparatus 3 generates a cross-sectional image (MRI image) indicating a state inside the body of a patient using a strong magnet and electromagnetic waves. The generated MRI image is associated with the patient information or the examination information (for example, recorded as header information of the image) and transmitted to the medical image server 5.

The X-ray imaging apparatus 4 is configured to include a hardware processor, a storage, an operation interface, a display, a communicator, an image generator, and the like. The image generator of the X-ray imaging apparatus 4 generates an X-ray image by emitting X-rays to a subject. The generated X-ray image is transmitted to the medical image server 5 or the image analyzer 6 in association with the patient information or the examination information.

The medical image server 5 is a computer apparatus including a hardware processor, a storage, an operation interface, a display, a communicator, and the like. The medical image server 5 has an image database (DB) 51 in the storage, and stores a medical image, such as an MRI image transmitted from the MRI apparatus 3 or an X-ray image transmitted from the X-ray imaging apparatus 4, in the image DB 51 so as to be associated with the patient information or the examination information. A medical image matching the search conditions requested by another apparatus included in the medical information processing system 100 is searched and read from the image DB 51 and transmitted to the requesting apparatus.

As the medical image server 5, a picture archiving and communication system (PACS) can be applied.

The image analyzer 6 is a computer apparatus including a hardware processor, a storage, an operation interface, a display, a communicator, and the like. The image analyzer 6 includes a deep learning unit 61 and a metal detection processor 62. The metal detection processor 62 performs metal detection processing on the X-ray image transmitted from the X-ray imaging apparatus 4 or the like using parameters learned by the deep learning unit 61, and stores detection result information regarding a metal contraindicated in MRI examinations (metal detection result information) in a detection result storage 63 so as to be associated with the patient information. Metal detection result information corresponding to the patient information transmitted from another apparatus included in the medical information processing system 100 is read from the detection result storage 63 and transmitted to the apparatus as a transmission source.

A structure containing a metal contraindicated in MRI examinations may be embedded in the body of the patient. Examples of the metal structure contraindicated in MRI examinations include an endoscope clip, a pacemaker, an inferior vena cava (IVC) filter, an indwelling catheter (ICD), an orthopedic implant, stent glasses, and SCS. However, the metal structure contraindicated in MRI examinations is not limited thereto.

[Operation of the Medical Information Processing System 100]

Next, an operation in the present embodiment will be described.

In the case of performing an examination using the MRI apparatus 3, it is checked in advance whether or not a metal contraindicated in MRI examinations is embedded in the body of the patient, and it is necessary to take measures, such as stopping the examination, when a metal contraindicated in the MRI examination is embedded.

In the present embodiment, an example will be described in which, when an X-ray image is captured, metal detection processing is performed on the captured X-ray image in advance by the image analyzer 6 and the metal detection result information is stored in the detection result storage 63 so as to be associated with the patient information, and when a laboratory technician or the like displays and checks the list (order list) of examination order information of the MRI examination target (here, the examination date is the next day) before the MRI examination (here, the day before the examination date), examination order information of a patient from whom a metal contraindicated in MRI examinations is detected is displayed in an identifiable manner.

(Metal Detection Processing at the Time of Capturing an X-Ray Image)

When the X-ray imaging apparatus 4 acquires an X-ray image by imaging, the hardware processor transmits the X-ray image to the medical image server 5 and the image analyzer 6 through the communicator.

In the image analyzer 6, when the X-ray image is received from the X-ray imaging apparatus 4, the hardware processor inputs the received X-ray image to the metal detection processor 62 to perform metal detection processing.

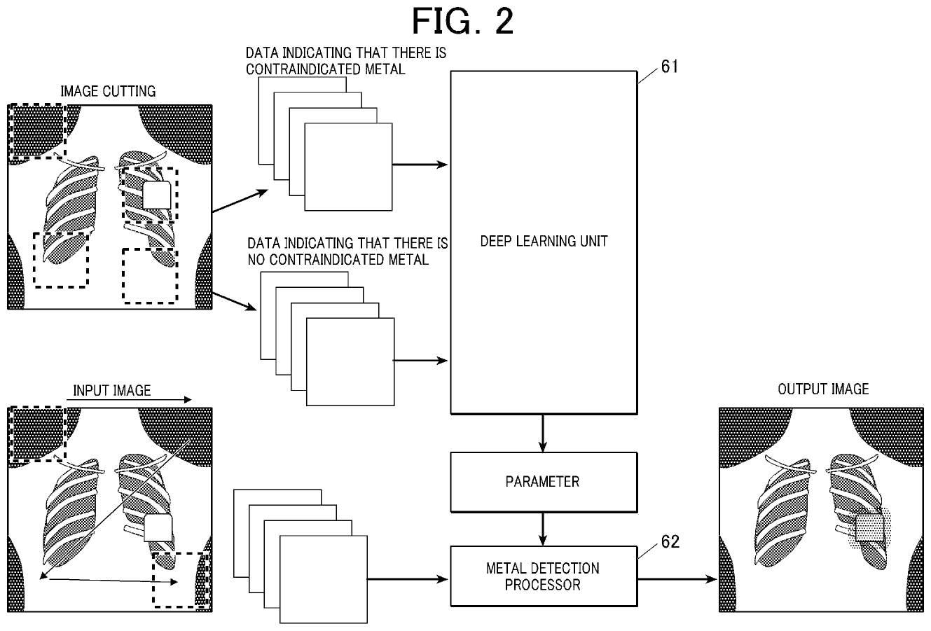

As illustrated in FIG. 2, the deep learning unit 61 and the metal detection processor 62 are provided in the image analyzer 6. The deep learning unit 61 performs machine learning using image data (data indicating that there is a contraindicated metal, data indicating that there is no contraindicated metal), which is a large number of image data cut out from a large number of X-ray images and in which the presence or absence of a metal contraindicated in MRI examinations is associated in advance, and creates parameters for performing metal detection processing by deep learning on the X-ray images input in the metal detection processor 62, and outputs the parameters to the metal detection processor 62. The metal detection processing is processing in which an X-ray image is cut out and a certainty factor as to whether or not a metal contraindicated in MRI examinations is included is calculated for each pixel, an image for which the maximum value of the certainty factor exceeds a predetermined threshold value is determined to include a metal contraindicated in MRI examinations (an image for which the maximum value of the certainty factor is equal to or less than the predetermined threshold value is determined to include no metal contraindicated in MRI examinations), and metal detection result information including the certainty factor (maximum value), the presence or absence of a metal contraindicated in MRI examinations, an image (referred to as a metal detection result image; for example, an image in which a detection position on the X-ray image is colored) illustrating the detection position (for example, a position where the certainty factor is greater than 0.5) of the MRI metal, and the like is output. The certainty factor takes a value of 0 to 1, and the certainty factor indicating that there is a contraindicated metal becomes higher as the value becomes closer to 1.

The metal detection processor 62 performs metal detection processing on the input X-ray image by deep learning using the parameters generated by the deep learning unit 61, and outputs metal detection result information.

As described above, since the metal detection processor 62 detects a metal contraindicated in MRI examinations from the X-ray image by machine learning using image data (data indicating that there is a contraindicated metal, data indicating that there is no contraindicated metal) in which the presence or absence of a metal contraindicated in MRI examinations is associated in advance, it is possible to accurately detect the metal contraindicated in MRI examinations. That is, it is possible to reduce omission of detection of a metal that is contraindicated in MRI examinations or to reduce erroneous detection of a metal that is not contraindicated in MRI examinations. For metals that are not contraindicated in MRI examinations, some types (materials) of metal are not contraindicated in MRI examinations, and some types of metal are contraindicated in MRI examinations but do not affect the MRI examinations (for example, a lead plate indicating the imaging direction or the reflection of the irradiation field aperture of the X-ray imaging apparatus 4).

The deep learning unit 61 and the metal detection processor 62 are realized by cooperation between the hardware processor of the image analyzer 6 and various learning programs stored in the storage, but may be configured by dedicated hardware.

In the image analyzer 6, when the metal detection processing ends, the hardware processor stores the metal detection result in the detection result storage 63 so as to be associated with the patient information.

(Image Check Before MRI Examination)

On a day before the MRI examination or the like, the laboratory technician inputs conditions for generating the order list of the MRI examination on the next day (examination order information indicating that the examination date is the next day and the modality is the MRI apparatus 3) using the operation interface in the medical information management apparatus 2, thereby giving an instruction (request) to generate the order list.

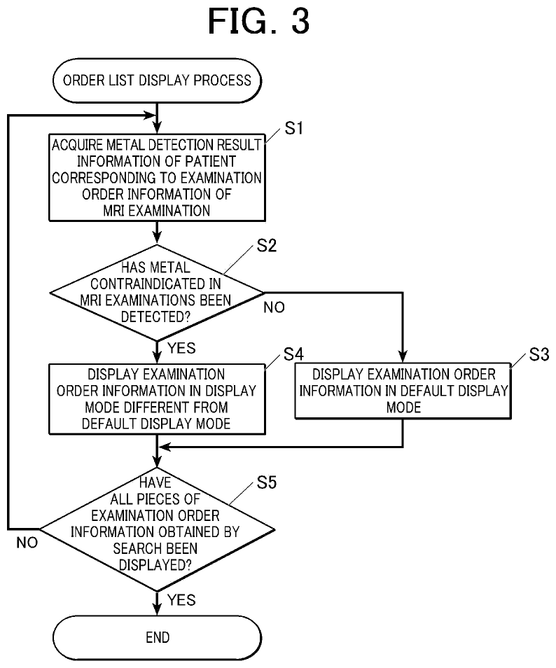

In the medical information management apparatus 2, when the instruction to generate the order list is given through the operation interface, the hardware processor searches for examination order information, which indicates that the examination date is the next day and the modality is the MRI apparatus 3, in the examination order information stored in the order DB 21. Then, the hardware processor of the medical information management apparatus 2 displays the order list by performing an order list display process illustrated in FIG. 3 on the examination order information obtained by the search.

Hereinafter, the order list display process will be described with reference to FIG. 3. The order list display process is realized by cooperation between the hardware processor of the medical information management apparatus 2 and a program stored in the storage.

First, the hardware processor of the medical information management apparatus 2 selects one piece of examination order information among the pieces of examination order information obtained by the search, makes a request for transmission of metal detection result information by transmitting patient information (for example, a patient ID) included in the selected examination order information to the image analyzer 6 through the communicator, and acquires metal detection result information of the patient corresponding to the examination order information (step S1).

Then, based on the received metal detection result information, the hardware processor determines whether or not a metal contraindicated in MRI examinations has been detected from the patient corresponding to the examination order information (step S2).

If it is determined that a metal contraindicated in MRI examinations has not been detected (step S2; NO), the hardware processor displays the examination order information in a default display mode (step S3), and the process proceeds to step S5.

In step S3, the hardware processor may perform control so as not to display the examination order information of the patient from whom a metal contraindicated in MRI examinations has not been detected.

If it is determined that a metal contraindicated in MRI examinations has been detected from the patient (step S2; YES), the hardware processor displays the examination order information in a display mode indicating that a metal contraindicated in MRI examinations has been detected, which is different from the default display mode (step S4), and the process proceeds to step S5.

In step S4, for example, display changes are made, such as displaying the examination order information in a predetermined color different from the default or displaying the examination order information with information of “There is a contraindicated metal” or the like added thereto. Therefore, the doctor or the laboratory technician who selects a patient from the examination order list and performs image check as to whether or not there is a contraindicated metal can easily determine on the list screen whether or not the patient is a target of actual visual image check.

In step S5, the hardware processor determines whether or not the processing of steps S1 to S4 has been completed for all the pieces of examination order information of the MRI examination obtained by the search (step S5).

If it is determined that the processing of steps S1 to S4 has not been completed for all the pieces of examination order information of the MRI examination obtained by the search (step S5; NO), the hardware processor returns to step S1 to repeat the processing of steps S1 to S4.

If it is determined that the processing of steps S1 to S4 has been completed for all the pieces of examination order information of the MRI examination obtained by the search (step S5; YES), the hardware processor ends the order display process.

When the list (order list) of the examination order information of the MRI examination on the next day is displayed on the display of the medical information management apparatus 2, the laboratory technician performs an image checking process.

In the image checking process, first, the laboratory technician selects examination order information (displayed in a display mode different from the default) of an image check target from the order list using the operation interface.

When the examination order information is selected from the order list through the operation interface, the hardware processor of the medical information management apparatus 2 causes the display to display a metal detection result image of the selected examination order information.

The laboratory technician checks the displayed metal detection result image. When it is checked that a metal contraindicated in MRI examinations is embedded, the laboratory technician performs an operation of adding, for example, information of “There is a contraindicated metal, stop the examination”, to the examination order information or giving an instruction to cancel the examination order. The hardware processor of the medical information management apparatus 2 may automatically generate a sentence to be added to the examination order information. For example, a sentence such as “There is a contraindicated metal in simple X-ray imaging on the chest taken on O (day), X (month)” or “There is no contraindicated metal in simple X-ray imaging on the chest taken on O (day), X (month)” is automatically generated and added to the examination order information according to the selection of the user. In this manner, a sentence to be added to the examination order information is automatically generated, so that it is possible to reduce the time and effort required for the laboratory technician to add information regarding the presence or absence of a contraindicated metal to the examination order information.

(At the Time of MRI Examination)

In the MRI apparatus 3, based on the operation of the laboratory technician or at the arrival of a predetermined time, the hardware processor requests the medical information management apparatus 2 to transmit examination order information of the MRI examination on the day and acquires the examination order information, and causes the display to display the order list of the MRI examination on the day.

The laboratory technician performs the MRI examination according to the displayed order list.

As described above, in the first embodiment, a metal that is contraindicated in MRI examinations is detected instead of detecting a metal region from the X-ray image. Therefore, it is possible to accurately detect whether or not a metal contraindicated in MRI examinations is embedded in the body of the patient. Specifically, since a metal contraindicated in MRI examinations is detected from the X-ray image by deep learning using parameters learned by machine learning using image data (data indicating that there is a contraindicated metal, data indicating that there is no contraindicated metal) in which the presence or absence of a metal contraindicated in MRI examinations is associated in advance, it is possible to accurately detect a metal contraindicated in MRI examinations. Therefore, it is possible to reduce the time and effort for image check required for the technician, the doctor, or the like to check whether or not a metal contraindicated in MRI examinations is embedded in the body of a patient, who is to be subjected to the MRI examination, using an image. When displaying the order list of the MRI examination, the examination order information of a patient from whom a metal contraindicated in MRI examinations has been detected is displayed in a mode different from those in other examinations. Therefore, since the laboratory technician or the like can easily recognize the examination order information of a patient from whom a metal contraindicated in MRI examinations has been detected and for whom image check is to be performed, it is possible to efficiently perform the image check.

(Modification Example of the First Embodiment)

The image check before the MRI examination may be performed by the MRI apparatus 3 immediately before the examination, not by the medical information management apparatus 2 on the previous day or the like. In this case, for example, the hardware processor of the MRI apparatus 3 acquires the examination order list of the MRI examination on the day, and then performs the same processing as the order list display process illustrated in FIG. 3 and displays the examination order information of a patient, from whom a metal contraindicated in MRI examinations has been detected, in a mode different from the default. When performing imaging based on the examination order information displayed in a mode different from the default, the laboratory technician displays the metal detection result image of the patient on the display of the MRI apparatus 3 and performs image check, so that it is possible to determine whether or not to perform the MRI examination.

### Second Embodiment

Hereinafter, a second embodiment of the present invention will be described.

In the second embodiment, metal detection processing is performed in advance on each X-ray image captured by the X-ray imaging apparatus 4, and the metal detection result information is stored. In the present embodiment, however, an example will be described in which, when examination order information of an MRI examination is generated, metal detection processing is performed on the X-ray image of a target patient of the generated examination order information.

(At the Time of X-Ray Imaging)

When the X-ray imaging apparatus 4 acquires an X-ray image by imaging, the hardware processor transmits the X-ray image to the medical image server 5 through the communicator.

In the medical image server 5, when the X-ray image is received through the communicator, the hardware processor stores the received X-ray image in the image DB 51 so as to be associated with the patient information and the examination information.

(Metal Detection Processing on an X-Ray Image of a Patient Corresponding to Generated Examination Order Information)

When the examination order information generated by the order generator 1 is received through the communicator of the medical information management apparatus 2, the hardware processor of the medical information management apparatus 2 determines whether or not the received examination order information is an examination using the MRI apparatus 3 as a modality. When it is determined that the received examination order information is an examination using the MRI apparatus 3 as a modality, the hardware processor transmits patient information of the received examination order information to the image analyzer 6 through the communicator to make a request for metal analysis processing.

In the image analyzer 6, when the patient information transmitted from the medical information management apparatus 2 is received through the communicator, the hardware processor requests the medical image server 5 to transmit an X-ray image of the received patient information through the communicator.

When the request to transmit the patient information and the X-ray image is received from the image analyzer 6, the hardware processor of the medical image server 5 reads an X-ray image of the patient information matching the received patient information from the image DB 51, and transmits the X-ray image to the image analyzer 6 through the communicator.

The image analyzer 6 performs the above-described metal detection processing on the X-ray image received from the medical image server 5, and stores the obtained metal detection result information in the detection result storage 63 so as to be associated with the patient information.

Since (Image check before MRI examination) is the same as that described in the first embodiment or the modification example, the description is cited.

In the second embodiment, since the metal detection processing is performed on the X-ray image of the patient corresponding to the generated examination order information of the MRI examination, the target of the metal detection processing can be limited as compared with the first embodiment. Therefore, it is possible to reduce the processing load on the image analyzer 6 or the storage capacity of the storage for storing the metal detection result information.

That is, in the second embodiment, in addition to the same effect as in the first embodiment, there is an effect that the processing efficiency can be further improved.

### Third Embodiment

Hereinafter, a third embodiment of the present invention will be described.

In the third embodiment, an example will be described in which metal detection processing is performed in advance on an X-ray image captured by the X-ray imaging apparatus 4 and the metal detection result information is stored and a doctor is prompted to check an image when the doctor requests the order generator 1 to generate examination order information of a patient from whom a metal contraindicated in MRI examinations has been detected.

Since the configuration in the third embodiment is the same as that described in the first embodiment, the description is cited, and the operation of the medical information processing system 100 in the third embodiment will be described.

Since (Metal detection processing at the time of X-ray imaging) is the same as that described in the first embodiment, the description is cited. That is, the X-ray image captured by the X-ray imaging apparatus 4 is subjected to metal detection processing in advance by the image analyzer 6, and the metal detection result information is stored in the detection result storage 63 so as to be associated with the patient information.

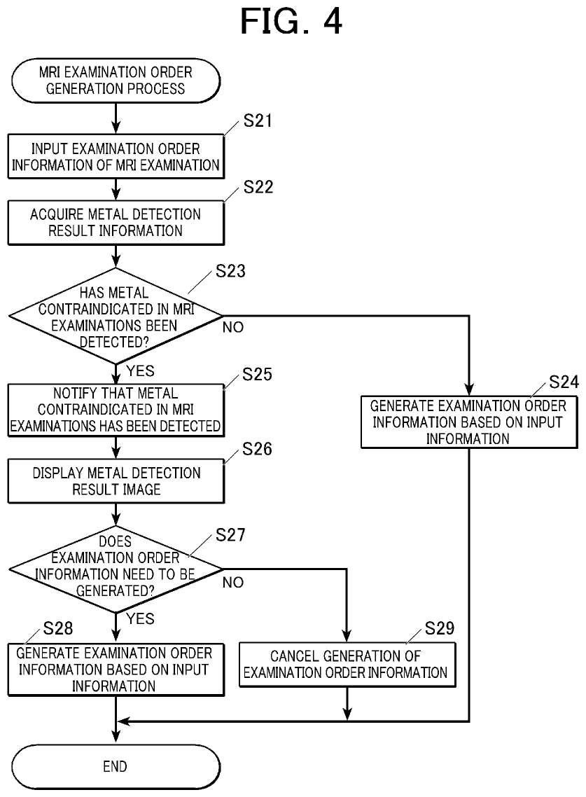

FIG. 4 is a flowchart illustrating an MRI examination order generation process performed by the hardware processor of the order generator 1. The MRI examination order generation process is performed by cooperation between the hardware processor of the order generator 1 and a program stored in the storage.

When the examination order information (examination order information indicating that the modality is the MRI apparatus 3) of the MRI examination is input through the operation interface and the generation instruction (request) is input (step S21), the hardware processor transmits patient information (for example, a patient ID) included in the input examination order information to the image analyzer 6 through the communicator to make a request for transmission of metal detection result information, and acquires metal detection result information of a patient corresponding to the input examination order information (step S22).

Then, based on the acquired metal detection result information, the hardware processor determines whether or not a metal contraindicated in MRI examinations has been detected from the patient corresponding to the input examination order information (step S23).

If it is determined that a metal contraindicated in MRI examinations has not been detected (step S23; NO), the hardware processor generates examination order information based on the input information and transmits the examination order information to the medical information management apparatus 2 (step S24), and ends the MRI order generation process.

If it is determined that a metal contraindicated in MRI examinations has been detected (step S23; YES), the hardware processor causes the display to display notification information for notifying that a metal contraindicated in MRI examinations has been detected from the X-ray image of the patient corresponding to the input examination order information (step S25).

In step S25, for example, a message such as “A metal contraindicated in MRI examinations has been detected from the patient. Please check the X-ray image”, an instruction button for displaying the metal detection result image, and the like are displayed on the display. Alternatively, when the order generator 1 includes a sound output unit, the above message may be output from the sound output unit. Alternatively, a predetermined warning sound or the like may be output.

When an instruction to display an image is given through the operation interface, the hardware processor displays the metal detection result image of the patient on the display (step S26), and proceeds to step S27.

The doctor observes the metal detection result image to check whether or not a metal contraindicated in MRI examinations is embedded in the patient, and gives an instruction again to generate examination order information based on the input through the operation interface when it is determined that a metal contraindicated in MRI examinations is not embedded in the patient (for example, when the detected metal is not contraindicated in MRI examinations). When it is determined that a metal contraindicated in MRI examinations is embedded, an instruction to cancel the generation of examination order information is given through the operation interface.

In step S27, the hardware processor determines whether or not an instruction to generate examination order information has been given again through the operation interface (step S27).

If it is determined that an instruction to generate examination order information has been given again through the operation interface (step S27; YES), the hardware processor generates the examination order information based on the input information and transmits the generated examination order information to the medical information management apparatus 2 through the communicator (step S28), and ends the MRI examination order generation process.

If it is determined that an instruction to cancel the generation of the examination order information has been given through the operation interface (step S27; NO), the hardware processor cancels the generation of the examination order information (step S29), and ends the MRI examination order generation process.

In the third embodiment, when a doctor tries to input and generate examination order information of a patient from whom a metal contraindicated in MRI examinations has been detected, the doctor can be notified of the fact and prompted to check the metal detection result image of the patient. When it is checked that a metal contraindicated in MRI examinations is embedded, the generation of examination order information of the MRI examination can be prevented. Therefore, unlike in the first and second embodiments, the technician does not need to check the X-ray image of a patient corresponding to the examination order information on a day before the examination or the like. It is possible to suppress useless examination order information generation or cancellation work.

That is, in the third embodiment, in addition to the same effect as in the first embodiment, the working efficiency can be further improved.

### Fourth Embodiment

Hereinafter, a fourth embodiment of the present invention will be described.

In the fourth embodiment, an example will be described in which metal detection processing is immediately performed on an X-ray image captured by the X-ray imaging apparatus 4 and a laboratory technician is prompted to check an X-ray image in which a metal contraindicated in MRI examinations is detected.

Since the configuration in the fourth embodiment is the same as that described in the first embodiment, the description is cited, and the operation of the medical information processing system 100 in the fourth embodiment will be described.

Hereinafter, an operation in the fourth embodiment will be described.

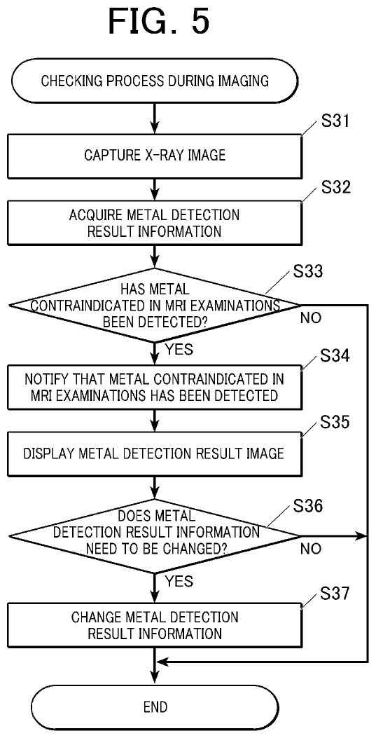

FIG. 5 is a flowchart illustrating a checking process during imaging that is performed by the hardware processor of the X-ray imaging apparatus 4. The checking process during imaging is performed by cooperation between the hardware processor of the X-ray imaging apparatus 4 and a program stored in the storage. It is assumed that examination order information of an X-ray examination is exported from the medical information management apparatus 2 to the X-ray imaging apparatus 4 and an order list is displayed on the display.

When examination order information is selected from the examination order information displayed on the display through the operation interface, the hardware processor of the X-ray imaging apparatus 4 performs X-ray imaging based on the selected examination order information to acquire an X-ray image (step S31). The acquired X-ray image is transmitted to the medical image server 5 through the communicator and stored therein.

Then, the hardware processor transmits the acquired X-ray image to the image analyzer 6 through the communicator, and causes the image analyzer 6 to perform metal detection processing to acquire metal detection result information (step S32).

Then, based on the received metal detection result information, the hardware processor determines whether or not a metal contraindicated in MRI examinations has been detected from the X-ray image (step S33).

If it is determined that a metal contraindicated in MRI examinations has not been detected from the X-ray image (step S33; NO), the hardware processor ends the checking process during imaging.

If it is determined that a metal contraindicated in MRI examinations has been detected from the X-ray image (step S33; YES), the hardware processor causes the display to display notification information for notifying that a metal contraindicated in MRI examinations has been detected from the patient (step S34).

In step S34, for example, a message such as “A metal contraindicated in MRI examinations has been detected from the patient. Please check the X-ray image”, an instruction button for displaying the metal detection result image, and the like are displayed on the display. Alternatively, when the X-ray imaging apparatus 4 includes a sound output unit, the above message may be output from the sound output unit. Alternatively, a predetermined warning sound or the like may be output.

When an instruction to display an image is given through the operation interface, the hardware processor displays the metal detection result image of the patient on the display (step S35), and proceeds to step S36.

For example, a “Confirm” button and a “Change” button are displayed on the display.

The doctor observes the metal detection result image, and presses the “Confirm” button using the operation interface when it is determined that a metal contraindicated in MRI examinations is included in the X-ray image. When it is determined that a metal contraindicated in MRI examinations is not included in the X-ray image, the doctor presses the “Change” button to give an instruction to change the metal detection result information to “No contraindicated metal”.

If an instruction to change the metal detection result information is given through the operation interface (step S36; YES), the hardware processor changes the metal detection result information, and transmits the changed metal detection result information to the image analyzer 6 through the communicator in accordance with the patient information to give an instruction for overwrite storage (step S37), and ends the checking process during imaging.

If an instruction indicating that the metal detection result information has been confirmed is given through the operation interface (step S36; NO), the hardware processor ends the checking process during imaging.

When the metal detection result information is received by the image analyzer 6, the received metal detection result information is overwritten and stored in the detection result storage 63.

In the fourth embodiment described above, the same effect as in the first embodiment can be obtained, and at the time of X-ray imaging, metal detection processing is performed on the captured X-ray image, and notification is provided to the laboratory technician on the spot so that the laboratory technician checks the situation when a metal contraindicated in MRI examinations is detected. Therefore, it is not necessary to check the image when generating the examination order information or when checking the order list on the day before the MRI examination.

That is, in the fourth embodiment, in addition to the same effect as in the first embodiment, the working efficiency can be further improved.

As described above, according to the medical information processing system 100, the metal detection processor 62 of the image analyzer 6 detects a metal contraindicated in MRI examinations from the X-ray image. Therefore, it is possible to accurately detect whether or not a metal contraindicated in MRI examinations is embedded in the body of the patient to be subjected to the MRI examination from the X-ray image, so that it is possible to reduce the time and effort required for the laboratory technician or the doctor to check the image. Since the detection of a metal contraindicated in MRI examinations from the X-ray image is performed by machine learning, it is possible to accurately detect a metal contraindicated in MRI examinations.

When displaying the list of examination order information of the MRI examination on the display, the hardware processor of the medical information management apparatus 2 according to each of the first and second embodiments displays examination order information regarding a patient corresponding to an X-ray image, in which a metal contraindicated in MRI examinations has been detected, and examination order information regarding a patient corresponding to an X-ray image, in which a metal contraindicated in MRI examinations has not been detected, in different display modes. Therefore, since a laboratory technician or a doctor can easily understand which examination order information patient needs image check, it is possible to reduce the time and effort for image check.

When a request to generate the examination order information of a patient corresponding to an X-ray image in which a metal contraindicated in MRI examinations has been detected is made, the hardware processor of the order generator 1 according to the third embodiment notifies that a metal contraindicated in MRI examinations has been detected from the medical image of the patient corresponding to the examination order information. Therefore, since it is possible to prompt to check whether or not the MRI examination can be performed before generating the examination order information, it is possible to prevent the generation of examination order information for a patient who cannot undergo the MRI examination. As a result, it is possible to omit image check for a patient corresponding to the generated examination order information. It is possible to suppress useless examination order information generation or cancellation work.

The description in each of the above embodiments is a preferable example of the present invention, and the present invention is not limited thereto.

For example, in the embodiments described above, since it is possible to accurately detect a metal contraindicated in MRI examinations, image check is performed only for the X-ray image of a patient from whom a metal contraindicated in MRI examinations has been detected. However, image check may be performed for X-ray images of all the patients to be subjected to the MRI examination.

For example, in the embodiments described above, the metal detection processing is performed using an X-ray image. However, the present invention is not limited to this. For example, medical images other than the MRI image, such as a CT image, may be used.

In the embodiments described above, the metal detection result information is stored in the image analyzer 6. However, the metal detection result information may be stored in the medical image server 5. Alternatively, after the image analyzer 6 performs the metal detection processing, a metal detection result image may be displayed on the display of the image analyzer 6 so that the doctor or the laboratory technician checks the image.

In the embodiments described above, the metal detection processing is performed by the image analyzer 6 that is an independent apparatus. However, any apparatus included in the medical information processing system 100 may have a function of the metal detection processing.

In the above description, an example is disclosed in which a hard disk, a semiconductor non-volatile memory, or the like is used as a computer-readable medium for a program according to the present invention, but the present invention is not limited to this example. As other computer-readable media, a portable recording medium, such as a CD-ROM, can be applied. A carrier wave is also applied as a medium for providing data of the program according to the present invention through a communication line.

The detailed configuration and detailed operation of each apparatus included in the medical information processing system can also be appropriately changed without departing from the spirit of the present invention.

In the above embodiments, since it is possible to accurately detect whether or not a metal contraindicated in MRI examinations is embedded in the body of a patient to be subjected to the MRI examination, it is possible to reduce the time and effort required for the laboratory technician or the doctor to check the image.

Although embodiments of the present invention have been described and illustrated in detail, the disclosed embodiments are made for purposes of illustration and example only and not limitation. The scope of the present invention should be interpreted by terms of the appended claims

What is claimed is:

1. A medical information processing system, comprising:
a first hardware processor that analyzes a medical image,
wherein the first hardware processor detects a metal contraindicated in MRI examinations from the medical image.

2. The medical information processing system according to claim 1, further comprising:
a display that displays a list of examination order information of an MRI examination; and
a second hardware processor that, when displaying the list of the examination order information on the display, displays examination order information regarding a patient corresponding to a medical image, in which a metal contraindicated in MRI examinations has been detected by the first hardware processor, and examination order information regarding a patient corresponding to a medical image, in which a metal contraindicated in MRI examinations has not been detected by the first hardware processor, in different display modes.

3. The medical information processing system according to claim 1, further comprising:
a third hardware processor that generates examination order information of an MRI examination,
wherein, when a request to generate examination order information of a patient corresponding to a medical image in which a metal contraindicated in MRI examinations has been detected by the first hardware processor is made, the third hardware processor notifies that a metal contraindicated in MRI examinations has been detected from the medical image of the patient corresponding to the examination order information.

4. The medical information processing system according to claim 3,
wherein the third hardware processor performs the notification by display or sound.

5. The medical information processing system according to claim 1,
wherein the first hardware processor detects a metal contraindicated in MRI examinations from the medical image by machine learning.
